(19) United States
(12) Patent Application Publication
(10) Pub. No.: US 2014/0048080 A1
(43) Pub. Date: Feb. 20, 2014

(54) ANATOMICAL EXTERNAL PRESSURE MANAGEMENT METHODS

(71) Applicants: Michael Dennis, Scappoose, OR (US); Casey Dennis, Sequim, OR (US); Christopher James, Portland, OR (US)

(72) Inventors: Michael Dennis, Scappoose, OR (US); Casey Dennis, Sequim, OR (US); Christopher James, Portland, OR (US)

(21) Appl. No.: 13/784,422

(22) Filed: Mar. 4, 2013

Related U.S. Application Data
(60) Provisional application No. 61/605,822, filed on Mar. 2, 2012.

(51) Int. Cl. A61F 5/32

(52) U.S. Cl. CPC A61F 5/32
USPC 128/889

(57) ABSTRACT

Methods for external anatomical pressure management are described, such as, methods for reducing the loss of lean body mass in a person experiencing anatomical support wherein the support contact pressure which is applied to the anatomy of the person is adjusted to below venous-return blood-flow-occluding pressure of the person.

### CROSS REFERENCE TO RELATED APPLICATIONS

This application claims the benefit of U.S. Provisional Application No. 61/605,822, filed on Mar. 2, 2012, which is hereby incorporated by reference for all purposes.

### BACKGROUND

The present disclosure relates generally to methods to manage the effects of continuous pressure on a body, such as may occur in bedridden patients, paralyzed people, or otherwise immobilized bodies. In particular, methods are described for reducing contact pressure between the body and a supporting structure such that the pressure on the body does not reach and exceed venous-return blood-flow-occluding pressure and thereby minimizes the loss of lean body mass.

Known methods of pressure management are not entirely satisfactory for the range of applications in which they are employed. For example, preventing pressure sores among bedridden elderly patients is a major concern for healthcare providers and caregivers. Pressure sores, also known as a pressure ulcer, bedsore, or decubitus ulcer, usually occur when patients are immobilized and confined to bed for a prolonged period of time. Unrelieved pressure on specific areas of the body can result in an injury that causes skin breakdown and an open sore.

Pressure sores can result from one period of sustained pressure or as a result of repeated incidents of blood flow interruption without adequate time for recovery. Pressure sores typically form over a bony area such as the back, tailbone, buttocks, hip, heels, elbows, and shoulders. Current prevention techniques include repositioning of the patients at timed intervals, using support surfaces to redistribute pressure, such as cushions, mattresses, beds, booties, and elbow pads, and keeping patients' skin clean and lubricated.

Thus, there exists a need for pressure management methods that improve upon and

advance known pressure management techniques. Examples of new and useful pressure management methods relevant to the needs existing in the field are discussed below.

### SUMMARY

The present disclosure is directed to methods for anatomical external pressure management. In certain embodiments, the inventive subject matter contemplates a method for reducing the loss of lean body mass in a person experiencing anatomical support wherein the support contact pressure which is applied to the anatomy of the person is adjusted to below venous-return blood-flow-occluding pressure of the person. In some embodiments, the support contact pressure may be reduced to below 0.6 psi. In other embodiments, the support contact pressure may be adjusted by providing a supporting surface which is applied to the anatomy of the person comprising a material having a return-pressure value below about 60% compression ranging between about 0.3 psi and about 0.5 psi.

The inventive subject matter is further directed to a method for eliminating support contact pressure, which is capable of occluding venous-return blood flow to a supported surface area of a person's anatomy, including supporting the surface area of the person's anatomy with a supporting structure including a compressible material providing a contact pressure between a supporting surface of the supporting structure and the supported surface area of the person's anatomy that is less than about 32 mm Hg. In some embodiments, the person may be immobilized or bedridden. In another possible embodiment, the compressible material may provide full-weight body support with no surface area of the person's anatomy having a contact pressure that exceeds 32 mm Hg. In some embodiments, the compressible material may be between about 20% and about 60% compressed in all positions substantially aligned with the supported surface area.

In further possible embodiments, the person's anatomy throughout the supported surface area may protrude into the supporting structure to compress the supporting structure at less than 60% of the support's original thickness. In some embodiments, the supporting structure may include a cushioning material, or a compressible foam material, for example a compressible viscoelastic foam such as a foam having a return-pressure versus deflection curve that remains substantially constant in the range of about 0.3 psi to about 0.5 psi. In some embodiments, the compressible viscoelastic foam may be selected from the group consisting of CONFOR® foam CF-40, CONFOR® foam CF-42, and #5010 CF Visco polyurethane Domfoam™.

In further possible embodiments, the compressible foam material may include a material having a return pressure that substantially remains within the range of about 15 mm Hg and about 25 mm Hg when the compressible foam material is compressed between about 20% and about 60%. In some embodiments, the compressible foam material may define a sufficient indentation force deflection to support the supported surface area of the person's anatomy while maintaining, at maximum, about 60% compression.

Further embodiments may include compressible foam material selected to produce a plurality of compression vectors directed toward substantially ail of the supported surface area of the person's anatomy and throughout the supported surface area as the person's anatomy protrudes into the compressible foam material, each of the compression vectors defining a contact pressure with a magnitude of less than about 32 mm Hg. For example, the compressible foam material may retain each of the compression vectors with a magnitude of less than about 32 mm Hg as the person's anatomy is supported by an indentation force deflection produced by the compressible foam when the compressible foam is at less than 60% compression across the supported surface area of the person's anatomy.

The inventive subject matter further contemplates a method for achieving enhanced

health in a person by recognizing that there is a relationship between the amount of mechanical pressure applied to the body of the person, apoptotic signaling of the person's cells, and resulting lean body mass loss of the person, and controlling the relationship between the amount of mechanical pressure applied and apoptotic signaling in a manner that is beneficial to the person by adjusting the amount of mechanical pressure applied to the body of the person so that the that the pressure remains below venous-return blood-flow-occluding pressure of the person. In the foregoing embodiment, the amount of mechanical pressure applied to the body may be provided by a compressible foam material defining a sufficient Indentation force deflection to support a supported surface area of the body of the person while maintaining, at maximum, about 60% compression.

### BRIEF DESCRIPTION OF THE DRAWINGS

FIG. 1 illustrates pressure profiles of different foam materials.

FIG. 2 is a cross-sectional view illustrating possible interaction between a portion of a body, a supporting structure, and a rigid base.

FIG. 3 is a cross-sectional view illustrating possible interaction between a portion of a body, a supporting structure, and a yielding base.

### DETAILED DESCRIPTION

The disclosed methods will become better understood through review of the following detailed description in conjunction with the figures. The detailed description and figures provide merely examples of the various inventions described herein. Those skilled in the art will understand that the disclosed examples may be varied, modified, and altered without departing from the scope of the inventions described herein. Many variations are contemplated for different applications and design considerations; however, for the sake of brevity, each and every contemplated variation is not individually described in the following detailed description,

Throughout the following detailed description, examples of various pressure management methods are provided. Related features in the examples may be identical, similar, or dissimilar in different examples. For the sake of brevity, related features will not be redundantly explained in each example. Instead, the use of related feature names will cue the reader that the feature with a related feature name may be similar to the related feature in an example explained previously. Features specific to a given example will be described in that particular example. The reader should understand that a given feature need not be the same or similar to the specific portrayal of a related feature in any given figure or example.

Some of the disclosed methods are based on the finding that the formation of decubitus ulcers has a relationship to conditions of externally applied anatomical pressure, which occurs at a level that reaches and exceeds that pressure which will cause occlusion of venous-return blood flow. In particular, pressure associated with such occlusion resides at around 32 mm Hg, which equates to approximately 0.6 psi.

Some methods are based on further findings that the onset of a decubitus ulcer condition, and the creation of an environment wherein the body can begin to heal an existing decubitus ulcer condition can be alleviated or aided by assuring that persons confined for periods of time effectively in non-motion support situations, such as people lying prone in a bed, as in a hospital or medical care facility, and persons spending long times in wheelchairs and the like, are supported, where possible, in a manner assuring that support contact pressure does not reach venous-return blood flow occlusion conditions. Additionally, methods may be based on a finding of a significant relationship between mechanical, body-support contact pressure, and (a) the onset of lean body mass (LBM) loss (significantly including muscle loss) and (b) related apoptotic signaling, which triggers potentially irreversible cell death. These findings have provided a basis for the pressure management methods of the present disclosure, which methods enable treatment and prevention of pressure sores.

Accordingly, some pressure management methods are aimed at eliminating, or at least minimizing, the loss of lean body mass with respect to long-term body support involves furnishing, as a support structure, a material which will confirmedly furnish full-weight body support, as required, with no area of the anatomy which is contacted in relation to that support experiencing a pressure that exceeds, or more preferably that even reaches, 32-mm Hg. While there may be many materials available that are capable of providing this kind of support, examples include a product known as CONFOR® foam CF-40, made by AEARO Specialty Composites in Indianapolis, Ind., USA, CONFOR® foam CF-42, also made by AEARO Specialty Composites, and a third product known as #5010 CF Visco Poly Domfoam™, made by Domfoam International, Inc., in Montreal, Quebec, Canada. Each of these viscoelastic foam products offers a return-pressure versus deflection characteristic curve which is characterized, with respect to compression up to, but not beyond, about 60% compression, with a return-pressure value, and consequently, a pressure application to a deflecting body, that remains substantially constant in the range of about 0.3- to about 0.5-psi, or a range of about 15.5- to about 25.9-mm Hg.

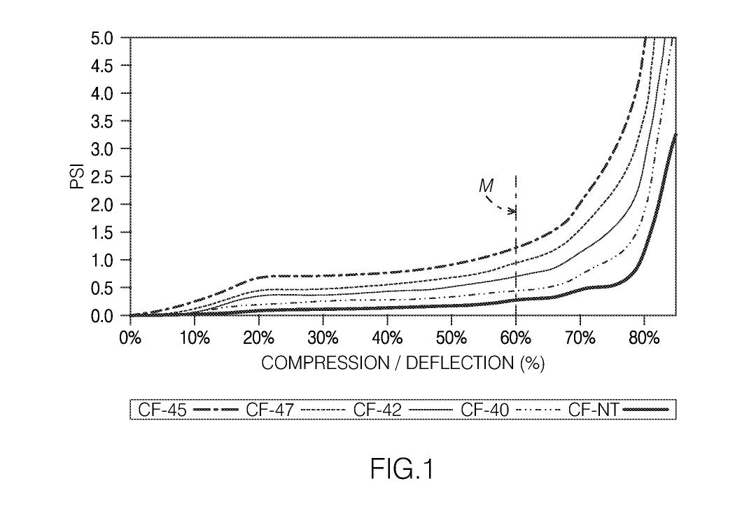

For example, FIG. 1 shows return-pressure profiles of CONFOR foam materials CF-45, CF-47, CF-42, CF-40, and CF-NT. The graph shows a compressive load, in psi units, applied to a supporting structure versus a percentage of compression/deflection that occurs in the supporting structure. A return-pressure curve having a lower or flatter profile below a maximum % compression M lies at about 60% compression for the materials shown, indicates good conformability of the material. Suitable foam materials define a sufficient indentation force deflection to support the supported surface area of the person's anatomy while maintaining, at maximum, about 60% compression.

Materials having such a profile offer the opportunity to furnish long-term anatomical support which may not produce a venous-return closure condition. With regard to a person's weight and the amount of body support contact area, supporting the body with materials having a relatively flat return-pressure profile as described above will not cause the supporting material to reach or exceed 60% compression.

Furthermore, providing a properly designed support may actually reduce or eliminate the loss of lean body mass (LBM), triggered by apoptotic signaling, which is historically associated with long-term, motion-limited anatomical support The disclosed methods recognize, and address, the situation that there exists a connection between LBM loss, pressure on the anatomy, and resultant apoptotic cell-destruction signaling.

According to one embodiment of the inventive subject matter, support may be provided by a product, such as the foam products mentioned above, in such a manner that, with regard to a person's body weight which is to be supported, and considering the body support contact area that will be involved, actual supporting will not produce compression of the supporting material at any location which exceeds or reaches 60% compression.

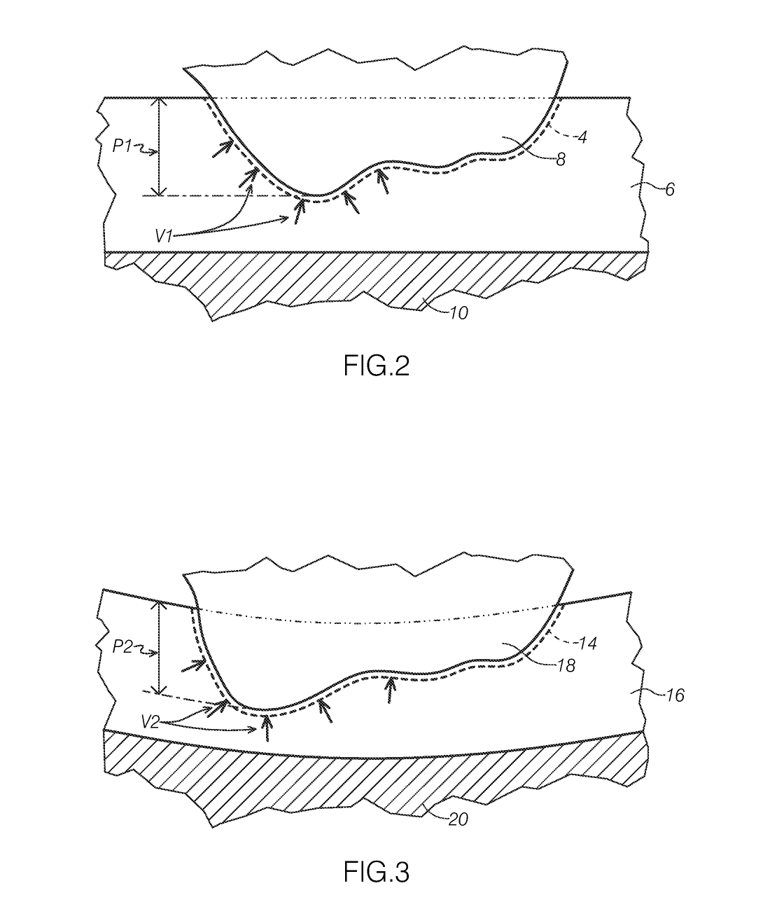

FIG. 2 and FIG. 3 illustrate possible interaction zones between a portion of a body, a supporting structure, for example made of a foam material, and a base. FIG. 2, shows a supporting structure 6, for example including a compressible foam material, producing a plurality of compression vectors V1 directed toward substantially all of a supported surface area 4 of the person's anatomy 8. Throughout the supported surface area 4, as the person's anatomy 8 protrudes into the supporting structure 6, each of the compression vectors V1 defines a contact pressure point with a magnitude of less than about 32 mm Hg. FIG. 2 shows vectors V1 when a supporting structure is carried by a rigid base 10. FIG. 3 shows a similar supporting structure 16 elected to produce a plurality of compression vectors V2 that are directed toward substantially all of a supported surface area 14 of the person's anatomy 18. However, a yielding base 20 is provided below supporting structure 16 leading to a curvature of supporting structure 16 and different contact pressure points indicated by vectors V2 along continuous support surface 14. Here too, according to an example embodiment of the inventive subject matter, the person's anatomy 18 is protruding into the supporting structure 16 and each of the compression vectors V2 defines a contact pressure point with a magnitude of less than about 32 mm Hg.

In the embodiments shown in FIG. 2 and FIG. 3, the respective compressible foam materials retain each of the compression vectors with a magnitude of less than about 32 mm Hg as the person's anatomy is supported by an indentation force deflection produced by the compressible foam when the compressible foam is at less than 60% compression across the supported surface area of the person's anatomy. A maximum compression of the supporting structure of less than about 60% is marked in FIGS. 2 and 3 and is referred to with the letters P1 and P2 respectively.

According to some embodiments of the inventive subject matter, the loss and/or restoration of a human patient Lean Body Mass (LBM) may be caused in part or whole by mechanical pressure against the patients tissue and or muscle when prone or seated or otherwise under the effect of gravity, but not necessarily in proximity to the pressure. In other words the body may respond to pressure at any location and consequently affect LBM. Furthermore, apoptosis signaling may occur to/from muscle and/or tissue cells and/or blood cells from the mechanical constriction of capillary functions and/or physical pressure on or about the intracellular and/or extracellular environments of such cells, whether from swelling, outside (the body tissue) weight, gravitational delivered g force, and/or and resistance to g force, etc.

Moreover, if was found that apoptosis signaling resulting from mechanical pressure

may affect oxygen processes in human ceils in intracellular or extracellular environments or otherwise in bodily processes. Products which support the human body with rebound pressures that are less than vascular closure pressure, to wit, under 0.6 psi, may have an effect on cellular health, oxygen dependent energy generation, such as ATP I ADP cycles and related systems, oxygen affected pathways, either downstream or upstream, and all related signaling. Furthermore, apotosis signaling and the effect on LBM may also affect gene transcription, up-regulation, down-regulation, protein synthesis, protein consumption and/or cleavage, cell death signaling and lesser included processes.

Previous studies have examined the loss of lean body mass, for example as it relates

to the supplementation of β-liydroxy-β-metbylbutyrate (HMB) in patients. See for example, Abbott Nutrition, 2010, p. 1-19, which is hereby incorporated by reference for all purposes. However, a connection between LBM loss and pressure on the body and resultant signaling has not previously been examined.

The inventive subject matter also relies on the finding that cell signaling triggered by various ionic parameters, electrolyte balances and alike could be affected by oxygen deprivations. Moreover, it was found that apoptotic signaling is one of the results of ceil apoptosis cascades and that such processes effected LBM generally, not localized to the points of impact or pressure. Patients are adversely impacted by pressure, as opposed to disease, as they became more inactive or immobilized. That condition, if alleviated would reduce their LBM losses. Furthermore, the greater the inflammation, the greater the benefit from pressure relief to any part

The disclosure above encompasses multiple distinct inventions with independent utility. While each of these inventions has been disclosed in a particular form, the specific embodiments disclosed and illustrated above are not to be considered in a limiting sense as numerous variations are possible. The subject matter of the Inventions includes all novel and non-obvious combinations and subcombinations of the various elements, features, functions and/or properties disclosed above and inherent to those skilled in the art pertaining to such inventions. Where the disclosure or subsequently filed claims recite “a” element, “a first” element, or any such equivalent term, the disclosure or claims should be understood to incorporate one or more such elements, neither requiring nor excluding two or more such elements.

Applicant(s) reserves the right to submit claims directed to combinations and subcombinations of the disclosed inventions that are believed to be novel and non-obvious. Inventions embodied in other combinations and subcombinations of features, functions, elements and/or properties may be claimed through amendment of those claims or presentation of new claims in the present application or in a related application. Such amended or new claims, whether they are directed to the same invention or a different invention and whether they are different, broader, narrower or equal in scope to the original claims, are to be considered within the subject matter of the inventions described herein.

The invention claimed is:

1. A method for reducing the loss of lean body mass in a person experiencing anatomical support, comprising adjusting the support contact pressure which is applied to the anatomy of the person to below venous-return blood-flow-occluding pressure of the person,

2. The method of claim 1, wherein the support contact pressure is reduced to below 0.6 psi.

3. The method of claim 1, wherein the support contact pressure is adjusted by providing a supporting surface, which is applied to the anatomy of the person comprising a material having a return-pressure value below about 60% compression ranging between about 0.3 psi and about 0.5 psi.

4. A method for reducing support contact pressure below a threshold prone to occlude venous-return blood flow to a supported surface area of a person's anatomy, comprising supporting the surface area of the person's anatomy with a supporting structure comprising a compressible material providing a contact pressure between a supporting surface of the supporting structure and the supported surface area of the person's anatomy that is less than about 32 mm Hg.

5. The method of claim 4, wherein the person is immobilized.

6. The method of claim 4, wherein the person is bedridden.

7. The method of claim 4, wherein the compressible material provides full-weight body support with no surface area of the person's anatomy having a contact pressure that exceeds 32 mm Hg.

8 The method of claim 4, wherein the compressible material is between about 20% and about 60% compressed in all positions substantially aligned with the supported surface area.

9. The method of claim 10, wherein the person's anatomy throughout the supported surface area protrudes into the supporting structure to compress the supporting structure at less than 60% of the support's original thickness.

10. The method of claim 4, wherein the supporting structure comprises a cushioning material.

11. The method of claim 4, wherein the supporting structure comprises a compressible foam material.

12. The method of claim 11, wherein the compressible foam material comprises a compressible viscoelastic foam.

13. The method of claim II, wherein the compressible foam material comprises a compressible viscoelastic foam having a return-pressure versus deflection curve that remains substantially constant in the range of about 0.3 psi to about 0.5 psi.

14. The method of claim 12, wherein the compressible viscoelastic foam is selected from the group consisting of CONFOR® foam CF-40, CONFOR® foam CF-42, and #5010 CF Visco polyurethane Domfoam™.

15. The method of claim 11, wherein the compressible foam material comprises a material having a return pressure that remains substantially constant within the range of about 15 mm Hg and about 25 mm Hg when the compressible foam material is compressed between about 20% and about 60%.

16. The method of claim 11, wherein the compressible foam material defines a sufficient indentation force deflection to support the supported surface area of the person's anatomy while maintaining, at maximum, about 60% compression.

17. The method of claim 11, wherein the compressible foam material is selected to produce a plurality of compression vectors directed toward substantially all of the supported surface area of the person's anatomy and throughout the supported surface area as the person's anatomy protrudes into the compressible foam material, each of the compression vectors defining a contact pressure with a magnitude of less than about 32 mm Hg.

18. The method of claim 17, wherein the compressible foam material retains each of the compression vectors with a magnitude of less than about 32 mm Hg as the person's anatomy is supported by an Indentation force deflection produced by the compressible foam when the compressible foam is at less than 60% compression across the supported surface area of the person's anatomy.

19. A method for achieving enhanced health in a person, comprising:
determining the relationship between the amount of mechanical pressure applied to the body of the person, apoptotic signaling of the person's cells in response to the amount of mechanical pressure applied, and resulting lean body mass loss of the person; and
controlling the relationship between the amount of mechanical pressure applied and apoptotic signaling in a manner that is beneficial to the person by adjusting the amount of mechanical pressure applied to the body of the person so that the that the pressure remains below venous-return blood-flow-occluding pressure of the person.

20. The method of claim 19, wherein the amount of mechanical pressure applied to the body is provided by a compressible foam material defining a sufficient indentation force deflection to support a supported surface area of the body of the person while maintaining, at maximum about 60% compression.
